(19) United States
(12) Patent Application Publication
(10) Pub. No.: US 2007/0281931 A1
(43) Pub. Date: Dec. 6, 2007

(54) Pharmaceutical Composition Containing a P2x7 Receptor Antagonist and Methotrexate

(76) Inventor: Nigel Boughton-Smith, Leicestershire (GB)

Correspondence Address: FISH & RICHARDSON P.C., P.O BOX 1022, MINNEAPOLIS, MN, 55440-1022, US

(21) Appl. No.: 10/558,322

(22) PCT Filed: May 27, 2004
(86) PCT No.: PCT/SE04/00815

§ 371 Date: Sep. 12, 2006

(30) Foreign Application Priority Data
May 29, 2003 (GB) 0312320.5
Jun. 5, 2003 (SE) 0301651-6

(51) Int. Cl. A61K 31/4985; A61K 31/537; A61P 19/02; A61P 29/00

(52) U.S. Cl. 514/230.5; 514/249

(57) ABSTRACT

The invention provides a pharmaceutical composition, pharmaceutical product or kit comprising a first active ingredient which is a P2X₇ receptor antagonist, and a second active ingredient which is N-[4-[[(2,4-diamino-6-pteridinyl)methyl]methylamino]benzoyl]-L-glutamic acid (methotrexate) or a pharmaceutically acceptable derivative thereof, for use in the treatment of inflammatory disorders.

The present invention relates to combinations of pharmaceutically active substances for use in the treatment of inflammatory conditions/disorders, especially rheumatoid arthritis.

Chronic inflammatory disorders such as rheumatoid arthritis are polygenic, highly complex, and involve multiple inflammatory and immune mechanisms. Treatment of these disorders has been largely empirical with a variety of therapeutic agents being used with little understanding of the mechanisms involved. Recent research suggests that two inflammatory mediators, the cytokines IL-1 and TNFalpha (TNFα), may play key roles in the inflammatory process in rheumatoid arthritis.

It would be desirable to develop new pharmaceuticals for use in treating inflammatory conditions/disorders.

In accordance with the present invention, there is therefore provided a pharmaceutical composition comprising, in admixture, a first active ingredient which is a P2X₇ receptor antagonist and which P2X₇ receptor antagonist is an adamantyl derivative, and a second active ingredient which is N-[4-[[(2,4-diamino-6-pteridinyl)methyl]methylamino]benzoyl]-L-glutamic acid (methotrexate) or a pharmaceutically acceptable derivative thereof.

The P2X₇ receptor (previously known as P2Z receptor) is a ligand-gated ion channel that is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, is known to lead, amongst other things, to the release of interleukin-1β (IL-1β).

An antagonist of the P2X₇ receptor is a compound or other substance that is capable of preventing, whether fully or partially, activation of the P2X₇ receptor.

Methods for assaying for P2X₇ receptor antagonism are known in the art, for example from WO 01/42194 which describes an assay based on the observation that when the P2X₇ receptor is activated using a receptor agonist in the presence of ethidium bromide (a fluorescent DNA probe), an increase in the fluorescence of intracellular DNA-bound ethidium bromide is observed. Thus, an increase in fluorescence can be used as a measure of P2X₇ receptor activation and therefore to quantify the effect of a compound or substance on the P2X₇ receptor.

In WO 01/42194, the assay is carried out by taking a 96-well flat bottomed microtitre plate and filling the wells with 250 μl of test solution comprising 200 μl of a suspension of THP-1 cells (2.5×10⁶ cells/ml) containing 10⁻⁴ M ethidium bromide, 25 μl of a high potassium buffer solution containing 10⁻⁵ M benzoylbenzoyl adenosine triphosphate (bbATP, a known P2X₇ receptor agonist), and 25 μl of the high potassium buffer solution containing 3×10⁻⁵ M test compound. The plate is covered with a plastics sheet and incubated at 37° C. for one hour. The plate is then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) are used separately in the test as controls. From the readings obtained, a pIC₅₀ figure is calculated for the test compound, this figure being the negative logarithm of the concentration of test compound necessary to reduce the bbATP agonist activity by 50%. A pIC₅₀ figure greater than 5.5 is normally indicative of an antagonist.

Examples of P2X₇ receptor antagonists which may be used in accordance with present invention include the compounds described in WO 00/61569, WO 01/42194, WO 01/44170 and WO 03/41707 the entire contents of which are incorporated herein by reference.

More specifically, in a first embodiment of the present invention the P2X₇ receptor antagonist is a compound of formula 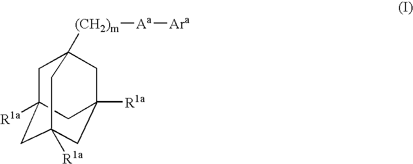
wherein m represents 1, 2 or 3;
each R^{1a} independently represents a hydrogen or halogen atom;
A^{a} represents C(O)NH or NHC(O);
Ar^{a} represents a group 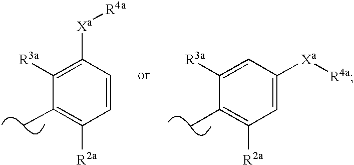
X^{a} represents a bond, an oxygen atom or a group CO, (CH₂)₁₋₆, CH═, (CH₂)₁₋₆O, O(CH₂)₁₋₆, O(CH₂)₂₋₆O, O(CH₂)₂₋₃O(CH₂)₁₋₃, CR′(OH), (CH₂)₁₋₃O(CH₂)₁₋₃, (CH₂)₁₋₃O(CH₂)₂₋₃O, NR^{5a}, (CH₂)₁₋₆NR^{5a}, NR^{5a}(CH₂)₁₋₆, (CH₂)₁₋₃NR^{5a}(CH₂)₁₋₃, O(CH₂)₂₋₆NR^{5a}, O(CH₂)₂₋₃NR^{5a}(CH₂)₁₋₃, (CH₂)₁₋₃NR^{5a}(CH₂)₂₋₃O, NR^{5a}(CH₂)₂₋₆O, NR^{5a}(CH₂)₂₋₃O(CH₂)₁₋₃, CONR^{5a}, NR^{5a}CO, S(O)ₙ, S(O)ₙCH₂, CH₂S(O)ₙ, SO₂NR^{5a} or NR^{5a}SO₂;
n is 0, 1 or 2;
R′ represents a hydrogen atom or a C₁-C₆ alkyl group;
one of R^{2a} and R^{3a} represents a halogen, cyano, nitro, amino, hydroxyl, or a group selected from (i) C₁-C₆ alkyl optionally substituted by at least one C₃-C₆ cycloalkyl, (ii) C₃-C₈ cycloalkyl, (iii) C₁-C₆ alkyloxy optionally substituted by at least one C₃-C₆ cycloalkyl, and (iv) C₃-C₈ cycloalkyloxy, each of these groups being optionally substituted by one or more fluorine atoms, and the other of R^{2a} and R^{3a} represents a hydrogen or halogen atom;
either R^{4a} represents a 3- to 9-membered saturated or unsaturated aliphatic heterocyclic ring system containing one or two nitrogen atoms and optionally an oxygen atom, the heterocyclic ring system being optionally substituted by one or more substituents independently selected from fluorine atoms, hydroxyl, carboxyl, cyano, C₁-C₆ alkyl, C₁-C₆ hydroxyalkyl, —NR^{6a}R^{7a}, —(CH₂)ᵣNR^{6a}R^{7a} and —CONR^{6a}R^{7a}, or R^{4a} represents a 3- to 8-membered saturated carbocyclic ring system substituted by one or more substituents independently selected from —NR^{6a}R^{7a}, —(CH₂)ᵣNR^{6a}R^{7a} and —CONR^{6a}R^{7a}, the ring system being optionally further substituted by one or more substituents independently selected from fluorine atoms, hydroxyl and C₁-C₆ alkyl;
r is 1, 2, 3, 4, 5 or 6;
R^{5a} represents a hydrogen atom or a C₁-C₆ alkyl or C₃-C₈ cycloalkyl group;
R^{6a} and R^{7a} each independently represent a hydrogen atom or a C₁-C₆ alkyl, C₂-C₆ hydroxyalkyl or C₃-C₈ cycloalkyl group, or R^{6a} and R^{7a} together with the nitrogen atom to which they are attached form a 3- to 8-membered saturated heterocyclic ring;
with the provisos that,
(a) when A^{a} represents C(O)NH and R^{4a} represents an unsubstituted 3- to 8-membered saturated aliphatic heterocyclic ring system containing one nitrogen atom, then X^{a} is other than a bond, and
(b) when A^{a} represents C(O)NH and X^{a} represents a group (CH₂)₁₋₆ or O(CH₂)₁₋₆, then R^{4a} does not represent an unsubstituted imidazolyl, unsubstituted morpholinyl, unsubstituted piperidinyl or unsubstituted pyrrolidinyl group, and
(c) when A^{a} represents NHC(O) and R^{4a} represents an unsubstituted 3- to 8-membered saturated aliphatic heterocyclic ring system containing one nitrogen atom, then X^{a} is other than a bond, and
(d) when A^{a} represents NHC(O) and X^{a} represents O(CH₂)₁₋₆, NH(CH₂)₁₋₆ or SCH₂, then R^{4a} does not represent an unsubstituted 1-piperidinyl or unsubstituted 1-pyrrolidinyl group, and
(e) when A^{a} represents NHC(O) and X^{a} represents O(CH₂)₂₋₃NH(CH₂)₂, then R^{4a} does not represent an imidazolyl group;
or a pharmaceutically acceptable salt or solvate thereof.

Compounds of formula (I) are described in WO 00/61569.

In a second embodiment of the present invention the P2X₇ receptor antagonist is a compound of formula 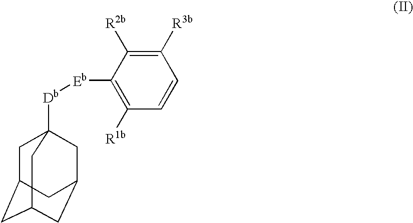
wherein D^{b} represents CH₂ or CH₂CH₂;
E^{b} represents C(O)NH or NHC(O);
R^{1b} and R^{2b} each independently represent a hydrogen or halogen atom, or an amino, nitro, C₁-C₆ alkyl or trifluoromethyl group;
R^{3b} represents a group of formula 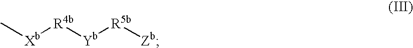
X^{b} represents an oxygen or sulphur atom or a group NH, SO or SO₂;
Y^{b} represents an oxygen or sulphur atom or a group NR^{11b}, SO or SO₂;
Z^{b} represents a group —OH, —SH, —CO₂H, C₁-C₆ alkoxy, C₁-C₆ alkylthio, C₁-C₆-alkylsulphinyl, C₁-C₆alkylsulphonyl, —NR^{6b}R^{7b}, —C(O)NR^{8b}R^{9b}, imidazolyl, 1-methylimidazolyl, —N(R^{10b})C(O)—C₁-C₆alkyl, C₁-C₆ alkylcarbonyloxy, C₁-C₆ alkoxycarbonyloxy, —OC(O)NR^{12b}R^{13b}, —OCH₂OC(O)R^{14b}, —OCH₂OC(O)OR^{15b} or —OC(O)OCH₂OR^{16b};
R^{4b} represents a C₂-C₆ alkyl group;
R^{5b} represents a C₁-C₆ alkyl group;
R^{6b}, R^{7b}, R^{8b}, R^{9b}, R^{10b}, R^{12b} and R^{13b} each independently represent a hydrogen atom, or a C₁-C₆ alkyl group optionally substituted by at least one hydroxyl group;
R^{11b} represents a hydrogen atom, or a C₁-C₆ alkyl group optionally substituted by at least one substituent independently selected from hydroxyl and C₁-C₆ alkoxy; and
R^{14b}, R^{15b} and R^{16b} each independently represent a C₁-C₆ alkyl group;
with the provisos that (i) when E^{b} represents NHC(O), X^{b} represents O, S or NH and Y^{b} represents O, then Z^{b} represents —NR^{6b}R^{7b} where R^{6b} represents a hydrogen atom and R^{7b} represents either a hydrogen atom or a C₁-C₆ alkyl group substituted by at least one hydroxyl group, and (ii) when E represents NHC(O), X^{b} represents O, S or NH, Y^{b} represents NH and R^{5b} represents CH₂CH₂, then Z^{b} is not —OH or imidazolyl;
or a pharmaceutically acceptable salt or solvate thereof.

Compounds of formula (II) are described in WO 01/42194.

In a third embodiment of the present invention the P2X₇ receptor antagonist is a compound of formula 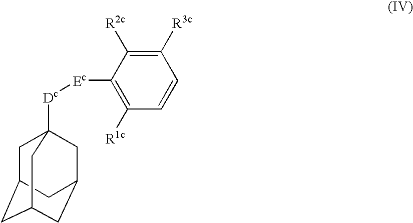
wherein D^{c} represents CH₂ or CH₂CH₂;
E^{c} represents C(O)NH or NHC(O);
R^{1c} and R^{2c} each independently represent hydrogen, halogen, amino, nitro, C₁-C₆ alkyl or trifluoromethyl, but R^{1c} and R^{2c} may not both simultaneously represent hydrogen;
R^{3c} represents a group of formula 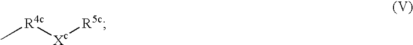
R^{4c} represents a C₁-C₆ alkyl group;
X^{c} represents an oxygen or sulphur atom or a group NR^{13c}, SO or SO₂;
R^{5c} represents hydrogen, or R^{5c} represents C₁-C₆ alkyl or C₂-C₆ alkenyl, each of which may be optionally substituted by at least one substituent selected from halogen, hydroxyl, (di)-C₁-C₆-alkylamino, —Y^{c}R^{6c}, 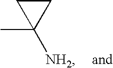
a 5- or 6-membered heteroaromatic ring comprising from 1 to 4 heteroatoms independently selected from nitrogen, oxygen and sulphur which heteroaromatic ring may itself be optionally substituted by at least one substituent selected from halogen, hydroxyl and C₁-C₆ alkyl;
Y^{c} represents an oxygen or sulphur atom or a group NH, SO or SO₂;
R^{6c} represents a group —R^{7c}Z^{c} where R^{7c} represents a C₂-C₆ alkyl group and Z^{c} represents an —OH, —CO₂H, —NR^{8c}R^{9c}, —C(O)NR^{10c}R^{11c} or —N(R^{12c})C(O)—C₁-C₆ alkyl group, and, in the case where Y^{c} represents an oxygen or sulphur atom or a group NH, R^{6c} additionally represents hydrogen, C₁-C₆ alkyl, C₁-C₆ alkylcarbonyl, C₁-C₆ alkoxycarbonyl, —C(O)NR^{14c}R^{15c}, —CH₂OC(O)R^{16c}, —CH₂C(O)OR^{17c} or —C(O)OCH₂OR^{18c};
R^{8c}, R^{9c}, R^{10c}, R^{11c} and R^{12c} each independently represent a hydrogen atom or a C₁-C₆ alkyl group;
R^{13c} represents hydrogen, C₃-C₈ cycloalkyl, C₃-C₈ cycloalkylmethyl, or R^{13c} represents a C₁-C₆ alkyl group optionally substituted by at least one substituent selected from hydroxyl and C₁-C₆ alkoxy; and
R^{14c}, R^{15c}, R^{16c}, R^{17c} and R^{18c} each independently represent a C₁-C₆ alkyl group;
with the proviso that when E^{c} is C(O)NH, X^{c} is O, NH or N(C₁-C₆ alkyl), then R^{5c} is other than a hydrogen atom or an unsubstituted C₁-C₆ alkyl group;
or a pharmaceutically acceptable salt or solvate thereof.

Preferred compounds of formula (IV) are those wherein R^{5c} represents an optionally substituted C₁-C₆ alkyl group, a preferred substituent being —Y^{c}—R^{6c}. When R^{5c} is substituted with a 5- or 6-membered heteroaromatic ring comprising from 1 to 4 heteroatoms, it is preferred that the number of heteroatoms in the ring is not greater than 2.

Compounds of formula (IV) are described in WO 01/44170.

In a fourth embodiment of the present invention the P2X₇ receptor antagonist is a compound of formula 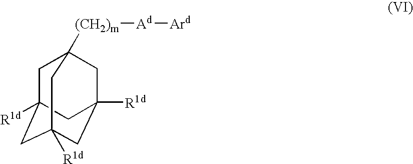
wherein m represents 1, 2 or 3;
each R^{1d} independently represents a hydrogen or halogen atom;
A^{d} represents C(O)NH or NHC(O);
Ar^{d} represents a group 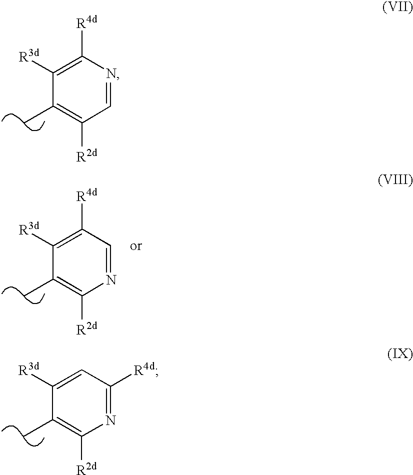
one of R^{2d} and R^{3d} represents halogen, nitro, amino, hydroxyl, or a group selected from (i) C₁-C₆ alkyl optionally substituted by at least one halogen atom, (ii) C₃-C₈ cycloalkyl, (iii) C₁-C₆ alkoxy optionally substituted by at least one halogen atom, and (iv) C₃-C₈ cycloalkyloxy, and the other of R^{2d} and R^{3d} represents a hydrogen or halogen atom;
R^{4d} represents a group 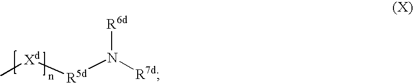
X^{d} represents an oxygen or sulphur atom or a group >N—R^{8d};
n is 0 or 1;
R^{5d} represents a C₁-C₅ alkyl group which may be optionally substituted by at least one substituent selected from hydroxyl, halogen and C₁-C₆ alkoxy;
R^{6d} and R^{7d} each independently represent a hydrogen atom, C₁-C₆ alkyl (optionally substituted by at least one substituent selected from hydroxyl, halogen, C₁-C₆ alkoxy, and (di)-C₁-C₄ alkylamino (itself optionally substituted by at least one hydroxyl group)), or C₃-C₈ cycloalkyl (optionally substituted by at least one substituent selected from hydroxyl, halogen and C₁-C₆ alkoxy); and
R^{8d} represents a hydrogen atom or a C₁-C₅ alkyl group which may be optionally substituted by at least one substituent selected from hydroxyl, halogen and C₁-C₆ alkoxy;
with the provisos that:
(a) when n is 0, then A^{d} is NHC(O), and
(b) when n is 1, X^{d} represents oxygen and A^{d} is C(O)NH, then R^{6d} and R^{7d} do not both simultaneously represent a hydrogen atom or do not both simultaneously represent an unsubstituted C₁-C₆ alkyl, or when one of R^{6d} and R^{7d} represents a hydrogen atom, then the other of R^{6d} and R^{7d} does not represent an unsubstituted C₁-C₆ alkyl; and
(c) when n is 1, X^{d} is oxygen, sulphur or >NH and A^{d} is NHC(O), then R^{6d} and R^{7d} do not both simultaneously represent a hydrogen atom or do not both simultaneously represent an unsubstituted C₁-C₆ alkyl, or when one of R^{6d} and R^{7d} represents a hydrogen atom, then the other of R^{6d} and R^{7d} does not represent an unsubstituted C₁-C₆ alkyl or —CH₂CH₂OH;
   or a pharmaceutically acceptable salt or solvate thereof.

Compounds of formula (VI) are described in WO 03/41707.

In another aspect of the present invention the P2X₇ receptor antagonist is a compound of formula 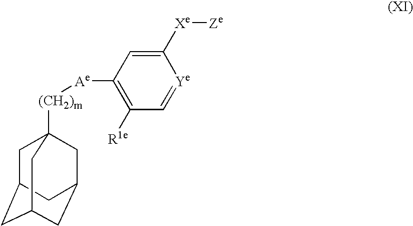
wherein m represents 1, 2 or 3;
A^{e} represents C(O)NH or NHC(O);
Y^{e} represents N or CH;
X^{e} represents a bond, CO, (CH₂)₁₋₆, O(CH₂)₁₋₆, (CH₂)₁₋₆NH(CH₂)₁₋₆, (CH₂)₁₋₆O(CH₂)₁₋₆, NH(CH₂)₁₋₆;
Z^{e} represents NR^{2e}R^{3e};
R^{1e} represents halogen, cyano, nitro, amino, hydroxyl, C₁-C₆ alkyl or C₃-C₈ cycloalkyl, which alkyl or cycloalkyl group can be optionally substituted by one or more fluorine atoms;
R^{2e} and R^{3e} each independently represent a hydrogen atom, C₁-C₆ alkyl or C₃-C₈ cycloalkyl, which alkyl or cycloalkyl group can be optionally substituted by one or more groups selected from hydroxyl, halogen or C₁-C₆ alkoxy,
or R^{2e} and R^{3e} together with the nitrogen atom to which they are attached form a 3- to 9-membered saturated mono- or bicyclic heterocyclic ring comprising from 1 to 2 nitrogen atoms and optionally an oxygen atom, which heterocyclic ring can be optionally substituted by one or more groups selected from hydroxyl, halogen or C₁-C₆ alkoxy;
or a pharmaceutically acceptable salt or solvate thereof.

Compounds of formula (XI) may be prepared by chemistry according or analogous to that described in the references cited herein above.

In a further aspect of the present invention the P2X₇ receptor antagonist is:—
2-Chloro-5-[[2-(2-hydroxy-ethylamino)-ethylamnio]-methyl]-N-(tricyclo[3.3.1.1^{3,7}]dec-1-ylmethyl)-benzamide,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethyl)ethyl]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-(piperidin-4-ylsulfinyl)-N-(tricyclo[3.3.1.1^{3,7}]dec-1-ylmethyl)-benzamide,
5-Chloro-2-[3-[(3-hydroxypropyl)amino]propyl]-N-(tricyclo[3.3.1.1^{3,7}]dec-1-ylmethyl)-4-pyridinecarboxamide,
2-Chloro-5-[3-[[(1R)-2-hydroxy-1-methylethyl]amino]propyl]-N-(tricyclo[3.3.1.1^{3,7}]dec-1-ylmethyl)-3-pyridinecarboxamide,
5-Chloro-2-[3-(ethylamino)propyl]-N-(tricyclo[3.3.1.1^{3,7}]dec-1-ylmethyl)-4-pyridinecarboxamide,
5-Chloro-2-[3-[(2-hydroxyethyl)amino]propyl]-N-(tricyclo[3.3.1.1^{3,7}]dec-1-ylmethyl)-4-pyridinecarboxamide,
5-Chloro-2-[3-[[(2S)-2-hydroxypropyl]amino]propyl]-N-(tricyclo[3.3.1.1^{3,7}]dec-1-ylmethyl)-4-pyridinecarboxamide,
N-[2-Methyl-5-(9-oxa-3,7-diazabicyclo[3.3.1]non-3-ylcarbonyl)phenyl]-tricyclo[3.3.1.1^{3,7}]decane-1-acetamide,

or a pharmaceutically acceptable salt or solvate of any one thereof.

Pharmaceutically acceptable salts include, where applicable, acid addition salts derived from pharmaceutically acceptable inorganic and organic acids such as a chloride, bromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, gluconate, tricarballylate, hydroxynaphthalene-carboxylate or oleate salt; and salts prepared from pharmaceutically acceptable inorganic and organic bases. Salts derived from inorganic bases include aluminium, ammonium, calcium, copper, ferric, ferrous, lithium, magnesium, manganic, manganous, potassium, sodium, zinc and bismuth salts. Particularly preferred are the ammonium, calcium, magnesium, potassium and sodium salts. Salts derived from pharmaceutically acceptable organic bases include salts of primary, secondary and tertiary amines, cyclic amines like arginine, betaine, choline and the like. Examples of pharmaceutically acceptable solvates include hydrates.

Examples of P2X₇ receptor antagonists that may conveniently be used in the present invention include:—
2-Chloro-5-[[2-(2-hydroxy-ethylamino)-ethylamino]-methyl]-N-(tricyclo[3.3.1.1^{3,7}]dec-1-ylmethyl)-benzamide, dihydrochloride
2-Chloro-5-[3-[(3-hydroxypropyl)amino]propyl]-N-(tricyclo[3.3.1.1]dec-1-ylmethyl)-benzamide, hydrochloride
(R)-2-Chloro-5-[3-[(2-hydroxy-1-methylethyl)amino]propyl]-N-(tricyclo[3.3.1.1^{3,7}]dec-1-ylmethyl)-benzamide, hydrochloride
2-Chloro-5-[[2-[(2-hydroxyethyl)amino]ethoxy]methyl]-N-(tricyclo[3.3.1.1^{3,7}]dec-1-ylmethyl)-benzamide, acetate (1:1) salt
2-Chloro-5-[3-[3-(methylamino)propoxy]propyl]-N-(tricyclo[3.3.1.1^{3,7}]dec-1-ylmethyl)benzamide, hydrochloride
2-Chloro-5-[3-(3-hydroxy-propylamino)-propoxy]-N-(tricyclo[3.3.1.1^{3,7}]dec-1-ylmethyl)-benzamide, hydrochloride
2-Chloro-5-[2-(3-hydroxypropylamino)ethylamino]-N-(tricyclo[3.3.1.1^{3,7}]dec-1-ylmethyl)-benzamide, acetate (1:1) salt
2-Chloro-5-[2-(3-hydroxypropylsulfonyl)ethoxy]-N-(tricyclo[3.3.1.1^{3,7}]dec-1-ylmethyl)-benzamide
2-Chloro-5-[2-[2-[(2-hydroxyethyl)amino]ethoxy]ethoxy]-N-(tricyclo[3.3.1.1^{3,7}]dec-1-ylmethyl)-benzamide, hydrochloride
2-Chloro-5-[[2-[[2-(1-methyl-1H-imidazolyl)ethyl]amino]ethyl]amino]-N-(tricyclo[3.3.1.1^{3,7}]dec-1-ylmethyl)-benzamide
2-Chloro-5-piperazin-1-ylmethyl-N-(tricyclo[3.3.1.1]dec-1-ylmethyl)-benzamide, dihydrochloride
2-Chloro-5-(4-piperidinyloxy)-N-(tricyclo[3.3.1.1^{3,7}]dec-1-ylmethyl)-benzamide, hydrochloride
2-Chloro-5-(2,5-diazabicyclo[2.2.1]hept-2-ylmethyl)-N-(tricyclo[3.3.1.1]dec-1-ylmethyl)-benzamide, hydrochloride
2-Chloro-5-(piperidin-4-ylsulfinyl)-N-(tricyclo[3.3.1.1^{3,7}]dec-1-ylmethyl)-benzamide
5-Chloro-2-[3-[(3-hydroxypropyl)amino]propyl]-N-(tricyclo[3.3.1.1^{3,7}]dec-1-ylmethyl)-4-pyridinecarboxamide,
2-Chloro-5-[3-[[(1R)-2-hydroxy-1-methylethyl]amino]propyl]-N-(tricyclo[3.3.1.1^{3,7}]dec-1-ylmethyl)-3-pyridinecarboxamide,
5-Chloro-2-[3-(ethylamino)propyl]-N-(tricyclo[3.3.1.1^{3,7}]dec-1-ylmethyl)-4-pyridinecarboxamide, hydrochloride
5-Chloro-2-[3-[(2-hydroxyethyl)amino]propyl]-N-(tricyclo[3.3.1.1^{3,7}]dec-1-ylmethyl)-4-pyridinecarboxamide, hydrochloride
5-Chloro-2-[3-[[(2S)-2-hydroxypropyl]amino]propyl]-N-(tricyclo[3.3.1.1^{3,7}]dec-1-ylmethyl)-4-pyridinecarboxamide, dihydrochloride, and
N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride.
N-[4-[[(2,4-diamino-6-pteridinyl)methyl]methylamino]benzoyl]-L-glutamic acid (methotrexate) has the following chemical structure:

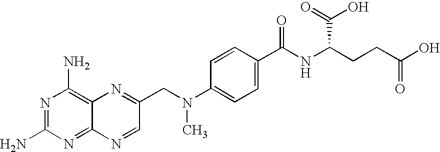

In the context of the present specification, unless otherwise stated, a pharmaceutically acceptable derivative of methotrexate means a pharmaceutically acceptable ester, salt or solvate of methotrexate or a pharmaceutically acceptable solvate of such an ester or salt.

Examples of suitable esters include lower alkyl (C₁-C₆ alkyl) esters.

Pharmaceutically acceptable salts include acid addition salts derived from pharmaceutically acceptable inorganic and organic acids such as a chloride, bromide, sulphate, phosphate, maleate, fumarate, tartrate, citrate, benzoate, 4-methoxybenzoate, 2- or 4-hydroxybenzoate, 4-chlorobenzoate, p-toluenesulphonate, methanesulphonate, ascorbate, acetate, succinate, lactate, glutarate, gluconate, tricarballylate, hydroxynaphthalene-carboxylate or oleate salt; and salts prepared from pharmaceutically acceptable inorganic and organic bases. Salts derived from inorganic bases include aluminium, ammonium, calcium, copper, ferric, ferrous, lithium, magnesium, manganic, manganous, potassium, sodium, zinc and bismuth salts. Particularly preferred are the ammonium, calcium, magnesium, potassium and sodium salts. Salts derived from pharmaceutically acceptable organic bases include salts of primary, secondary and tertiary amines, cyclic amines like arginine, betaine, choline and the like.

Examples of pharmaceutically acceptable solvates include hydrates.

The preparation of methotrexate is described, for example, by Seeger et al., J. Am. Chem. Soc., 71, 1753 (1949). Pharmaceutically acceptable derivatives of methotrexate may be prepared by methods conventional in the art.

Presently available oral and intravenous formulations of methotrexate include “Rheumatrex” (trade mark) methotrexate dose pack (Lederle Laboratories, Wayne, N.J., USA); methotrexate tablets (Mylan Pharmaceuticals Inc., Morgantown, W.V., USA; Roxane Laboratories, Inc., Columbus, Ohio, USA); methotrexate sodium tablets for injection (Immunex Corporation, Seattle, Wash., USA); and “methotrexate LPF” (trade mark) sodium injection (Immunex Corporation, Seattle, Wash., USA). Methotrexate is also commercially available from Pharmacochemie (Netherlands).

The active ingredients used in the present invention may be capable of existing in stereoisomeric forms. It will be understood that the invention encompasses all geometric and optical isomers of the active ingredients and mixtures thereof including racemates. Tautomers and mixtures thereof also form an aspect of the present invention.

The invention also provides a pharmaceutical product comprising, in combination, a preparation of a first active ingredient which is a P2X₇ receptor antagonist and which P2X₇ receptor antagonist is an adamantyl derivative, and a preparation of a second active ingredient which is N-[4-[[(2,4-diamino-6-pteridinyl)methyl]methylamino]benzoyl]-L-glutamic acid (methotrexate) or a pharmaceutically acceptable derivative thereof, for simultaneous, sequential or separate use in therapy.

In another aspect, the invention provides a kit comprising a preparation of a first active ingredient which is a P2X₇ receptor antagonist and which P2X₇ receptor antagonist is an adamantyl derivative, a preparation of a second active ingredient which is N-[4-[[(2,4-diamino-6-pteridinyl)methyl]methylamino]benzoyl]-L-glutamic acid (methotrexate) or a pharmaceutically acceptable derivative thereof, and instructions for the simultaneous, sequential or separate administration of the preparations to a patient in need thereof.

It has been found that the choice of active ingredients according to the invention is advantageous because it results in a beneficial anti-inflammatory effect and, accordingly, can be used to treat various acute and chronic inflammatory conditions/disorders such as rheumatoid arthritis.

The pharmaceutical composition of the invention may be prepared by mixing the first active ingredient with the second active ingredient. Therefore, in a further aspect of the present invention, there is provided a process for the preparation of a pharmaceutical composition which comprises mixing a first active ingredient which is a P2X₇ receptor antagonist and which P2X₇ receptor antagonist is an adamantyl derivative, with a second active ingredient which is N-[4-[[(2,4-diamino-6-pteridinyl)methyl]methylamino]benzoyl]-L-glutamic acid (methotrexate) or a pharmaceutically acceptable derivative thereof.

The first and second active ingredients may alternatively be administered simultaneously (other than in admixture as described above), sequentially or separately to treat inflammatory conditions. By sequential is meant that the first and second active ingredients are administered, in any order, one immediately after the other. They still have the desired effect if they are administered separately but less than about 4 hours apart, preferably less than about 2 hours apart, more preferably less than about 30 minutes apart.

The first and second active ingredients are conveniently administered by oral or parenteral (intraarticular or inhaled) administration using conventional systemic dosage forms, such as tablets, capsules, pills, powders, aqueous or oily solutions or suspensions, emulsions and sterile injectable aqueous or oily solutions or suspensions. These dosage forms will usually include one or more pharmaceutically acceptable ingredients which may be selected, for example, from adjuvants, carriers, binders, lubricants, diluents, stabilising agents, buffering agents, emulsifying agents, viscosity-regulating agents, surfactants, preservatives, flavourings and colorants.

Oral administration is preferred.

For the above-mentioned therapeutic uses the dosages administered will, of course, vary with the first and second active ingredients employed, the mode of administration, the treatment desired and the condition or disorder indicated. However, in general, satisfactory results will be obtained when the total, combined, daily dosage of first and second active ingredients, when taken orally, is in the range from 10 to 2000 milligrammes (mg), particularly from 10, 20, 30, 40, 50, 100, 150, 200 or 300 to 1800, 1500, 1200, 1000, 800, 700, 600, 500 or 400 mg.

The pharmaceutical composition, pharmaceutical product or kit according to the invention may be administered as divided doses from 1 to 4 times a day, and preferably once or twice a day.

The present invention further provides the use of a pharmaceutical composition, pharmaceutical product or kit according to the invention in the manufacture of a medicament for the treatment of an inflammatory disorder.

Also, the present invention provides a method of treating an inflammatory disorder which comprises administering a therapeutically effective amount of a pharmaceutical composition of the invention to a patient in need thereof.

Still further, the present invention provides a method of treating an inflammatory disorder which comprises simultaneously, sequentially or separately administering:

(a) a (therapeutically effective) dose of a first active ingredient which is a P2X₇ receptor antagonist and which P2X₇ receptor antagonist is an adamantyl derivative; and

(b) a (therapeutically effective) dose of a second active ingredient which is N-[4-[[(2,4-diamino-6-pteridinyl)methyl]methylamino]benzoyl]-L-glutamic acid (methotrexate) or a pharmaceutically acceptable derivative thereof,

to a patient in need thereof.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the condition or disorder in question. Persons at risk of developing a particular condition or disorder generally include those having a family history of the condition or disorder, or those who have been identified by genetic testing or screening to be particularly susceptible to developing the condition or disorder.

The invention further relates to triple combination therapies for the treatment of any one of rheumatoid arthritis, osteoarthritis, osteoporosis, psoriasis, inflammatory bowel diseases, COPD, asthma, allergic rhinitis or cancer or the neurodegenerative diseases such as multiple sclerosis, Alzheimer's disease or stroke.

For the treatment of rheumatoid arthritis, the pharmaceutical composition of the invention may be combined with “biological agents” such as IL-1 receptor antagonists (e.g. Anakinra) and IL-1 trap, IL-18 receptor, anti-IL-6 Ab, anti-CD20 Ab, anti-IL-15 Ab and CTLA4Ig.

Suitable agents to be used in combination with the pharmaceutical composition of the invention include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin. Cylco-oxygenase inhibiting nitric oxide donors (CINOD's) and “disease modifying agents” (DMARDs) such as cyclosporine A, leflunomide; ciclesonide; hydroxychloroquine, d-penicillamine, auranofin or parenteral or oral gold may also be used.

The present invention still further relates to the combination of a pharmaceutical composition of the invention together with a leukotriene biosynthesis inhibitor, 5-lipoxygenase (5-LO) inhibitor or 5-lipoxygenase activating protein (FLAP) antagonist selected from the group consisting of zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2n cyanonaphthalene compounds such as L-739,010; 2-cyanoquinoline compounds such as L-746,530; indole and quinoline compounds such as MK-591, MK-886, and BAY×1005.

The present invention still further relates to a pharmaceutical composition of the invention together with a receptor antagonist for leukotrienes LTB₄, LTC₄, LTD₄, and LTE₄ selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to a pharmaceutical composition of the invention together with a PDE4 inhibitor including inhibitors of the isoform PDE4D.

The present invention still further relates to a pharmaceutical composition of the invention together with a antihistaminic H₁ receptor antagonists including cetirizine, loratadine, desloratadine, fexofenadine, astemizole, azelastine, and chlorpheniramine.

The present invention still further relates to a pharmaceutical composition of the invention together with a gastroprotective H₂ receptor antagonist or the proton pump inhibitors (such as omeprazole)

The present invention still further relates to a pharmaceutical composition of the invention together with an α₁- and α₂-adrenoceptor agonist vasoconstrictor sympathomimetic agent, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to a pharmaceutical composition of the invention together with anticholinergic agents including ipratropium bromide; tiotropium bromide; oxitropium bromide; pirenzepine; and telenzepine.

The present invention still further relates to a pharmaceutical composition of the invention together with methylxanthanines including theophylline and aminophylline; sodium cromoglycate; or muscarinic receptor (M1, M2, and M3) antagonist.

The present invention still further relates to a pharmaceutical composition of the invention together with a modulators of chemokine receptor function such as CCR1, CCR2, CCR2A, CCR2B, CCR3, CCR4, CCR5, CCR6, CCR7; CCR8, CCR9, CCR10 and CCR11 (for the C—C family); CXCR1, CXCR3, CXCR4 and CXCR5 (for the C—X—C family) and CX₃CR1 for the C—X₃—C family.

The present invention still further relates to a pharmaceutical composition of the invention together with an insulin-like growth factor type I (IGF-1) mimetic.

The present invention still further relates to a pharmaceutical composition of the invention together with (a) tryptase inhibitors; (b) platelet activating factor (PAF) antagonists; (c) interleukin converting enzyme (ICE) inhibitors; (d) IMPDH inhibitors; (e) adhesion molecule inhibitors including VLA-4 antagonists; (f) cathepsins; (g) glucose-6 phosphate dehydrogenase inhibitors; (h) kinin-B₁- and B₂-receptor antagonists; (i) anti-gout agents, e.g., colchicine; (j) xanthine oxidase inhibitors, e.g., allopurinol; (k) uricosuric agents, e.g., probenecid, sulfinpyrazone, and benzbromarone; (l) growth hormone secretagogues; (m) transforming growth factor (TGFβ); (n) platelet-derived growth factor (PDGF); (o) fibroblast growth factor, e.g., basic fibroblast growth factor (bFGF); (p) granulocyte macrophage colony stimulating factor (GM-CSF); (q) capsaicin cream; (r) Tachykinin NK₁ and NK₃ receptor antagonists selected from the group consisting of NKP-608C; SB-233412 (talnetant); and D-4418; and (s) elastase inhibitors selected from the group consisting of UT-77 and ZD-0892 (t) induced nitric oxide synthase inhibitors (iNOS) or (u) chemoattractant receptor-homologous molecule expressed on TH2 cells, (CRTH2 antagonists).

The pharmaceutical composition of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, induced nitric oxide synthase inhibitors (iNOS inhibitors), and the cyclo-oxygenase inhibiting nitric oxide donors (CINOD's) analgesics (such as paracetamol and trarnadol), cartilage sparing agents such as diacerein, doxycyline and glucosamine, and hyaluronic acids such as hyalgan and synvisc.

The pharmaceutical composition of the invention may also be used in combination with existing therapeutic agents for the treatment of inflammatory bowel diseases (Ulcerative colitis and Crohn's disease). Suitable agents to be used include 5-amino-salicylates, the thiopurines, azathioprine and 6-mecaptorurine.

The pharmaceutical composition of the invention may also be used in combination with anticancer agents such as endostatin and angiostatin or cytotoxic drugs such as adriamycin, daunomycin, cis-platinum, etoposide, taxol, taxotere and farnesyl transferase inhibitors, VegF inhibitors, and antimetabolites such as antineoplastic agents, especially antimitotic drugs including the vinca alkaloids such as vinblastine and vincristine.

The pharmaceutical composition of the invention may also be used in combination with antiviral agents such as Viracept, AZT, aciclovir and famciclovir, and antisepsis compounds such as Valant.

The pharmaceutical composition of the invention may also be used in combination with calcium channel blockers, lipid lowering agents such fibrates, beta-blockers, Ace inhibitors, Angiotensin-2 receptor antagonists and platelet aggregation inhibitors.

The pharmaceutical composition of the invention may also be used in combination with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti Alzheimer's drugs such as donepezil, tacrine, propentofylline or metrifonate.

The pharmaceutical composition of the invention may also be used in combination with osteoporosis agents such as roloxifene, droloxifene, lasofoxifene or fosomax and immunosuppressant agents such as FK-506, rapamycin, cyclosporine, and azathioprine.

The present invention will now be further understood by reference to the following illustrative examples.

The following P2X₇ antagonists were employed in the examples:—

### 1. N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride

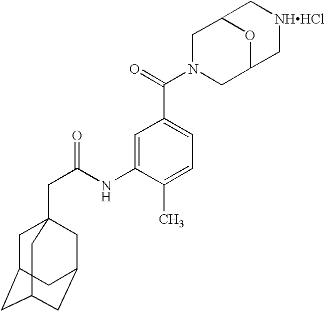

P2X₇ antagonist 1. (N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride) was prepared as follows.

### a) 3-(4-Methyl-3-nitrobenzoyl)-7-(phenylmethyl)-9-oxa-3,7-diazabicyclo[3.3.1]nonane

Oxalyl chloride (9.6 ml) in dichloromethane (30 ml) was added dropwise over 45 minutes to an ice-cooled solution of 4-methyl-3-nitro-benzoic acid (10.0 g) in dichloromethane (320 ml) containing DMF (0.1 ml). The reaction mixture was stirred at room temperature for 1 hour then concentrated in vacuo. The acid chloride was taken into THF (320 ml) and cooled in an ice-bath before adding N,N-diisopropylethylamine (38 ml) then 3-(phenylmethyl)-9-oxa-3,7-diazabicyclo[3.3.1]nonane, dihydrochloride (16.0 g) (prepared as described in WO 01/028992) portionwise. The reaction was stirred for 18 hours then diluted with ethyl acetate (600 ml) and washed with water (2×200 ml) and saturated sodium bicarbonate (aq) (3×150 ml) then dried (MgSO₄), filtered and concentrated to afford the sub-titled compound (18.5 g).

m/z=382

### b) 3-(3-Amino-4-methylbenzoyl)-7-(phenylmethyl)-9-oxa-3,7-diazabicyclo[3.3.1]nonane

Reduced iron powder (7.9 g) was added over 15 minutes to a stirred solution of the product of step a) (18.0 g) and ammonium chloride (7.5 g) in ethanol/water (3:1, 320 ml) at 70° C. The reaction mixture was heated at reflux for 2 hours then filtered and concentrated in vacuo. The residue was taken into ethyl acetate (400 ml), washed with water (2×150 ml) then the organic phase dried (MgSO₄) and concentrated in vacuo to afford the sub-title compound (14.5 g).

m/z=352

### c) N-[2-Methyl-5-[[7-(phenylmethyl)-9-oxa-3,7-diazabicyclo[3.3.1]non-3-yl]carbonyl]phenyl]-tricyclo[3.3.1.1^{3,7}]decane-1-acetamide

Prepared by the method of step a) using 1-adamantaneacetic acid and the product of step b). Recrystallisation (ethyl acetate) afforded the sub-title compound.

m/z 528

### d) N-[2-Methyl-5-(9-oxa-3,7-diazabicyclo[3.3.1]non-3-ylcarbonyl)phenyl]-tricyclo[3.3.1.1^{3,7}]decane-1-acetamide, hydrochloride

4M HCl in 1,4-dioxane (8 ml) was added to a solution of the product of step c) (13.0 g) in ethyl acetate (300 ml). The resulting precipitate was isolated by filtration then suspended in ethanol (300 ml) and 5% palladium on carbon (1.2 g) added. The reaction mixture was stirred under 3 atmospheres pressure of hydrogen for 36 hours. Methanol was then added under an atmosphere of nitrogen, then the catalyst removed by filtration and the filtrate concentrated in vacuo. Recrystallisation (isopropanol:methanol 25:1, 800 ml) gave the title compound (9.1 g).

m/z 438 (M+H)⁺

δ_{H} (400 MHz, d₆-DMSO, Me₄Si, 90° C.) 9.06 (1H, s), 7.64 (1H, s), 7.25 (1H, m), 7.19 (1H, m), 4.15 (2H, s), 3.96 (2H, d, J 14 Hz), 3.35-3.23 (6H, m), 2.26 (3H, s), 2.14 (2H, s), 1.96 (3H, br s), 1.69-1.62 (12H, m).

### EXAMPLE 1

### Pharmacological Analysis to Determine the Effect of Methotrexate P2X₇ Antagonist Combinations (Without Addition of a P2X₇ Agonist)

Human peripheral blood monocytes were prepared from the blood of healthy human volunteers collected in EDTA blood tubes. Monocytes were isolated by serial gradient centrifugation and washing to produce a pure population of cells. Lipopolysacharide (LPS) was then added to the cell suspension in tissue culture and this was incubated for 4-12 hours at 37 degrees centigrade. Methotrexate and/or a P2X₇ antagonist or vehicle was then added to the cells. After incubation, samples of cell supernatants were transferred to a 96-well plate for subsequent cytokine and mediator measurements. The formation of inflammatory mediators was measured in the cell supernatants by specific ELISA assays for the cytokines IL-1, IL-18, TNFα and for other mediators including PGE2, NO and matrix metalloproteinases (MMPs). The levels of mediators released in the presence of a P2X₇ receptor antagonist alone, or in the presence of methotrexate alone, or in the presence of a combination of a P2X₇ receptor antagonist with methotrexate were determined. The effects of the antagonists/methotrexate alone and in combination were then compared. Statistically significant levels of inhibitory activity against a single mediator (IL-1 or TNFα) or on multiple mediators by P2X₇ antagonist/methotrexate combinations, in comparison to that achieved by either a P2X₇ antagonist or methotrexate alone, is an indicator for increased efficacy in the treatment of disease.

### EXAMPLE 2

### Pharmacological Analysis to Determine the Effect of Methotrexate P2X₇ Antagonist Combinations (With Addition of a P2X₇ Agonist)

Human peripheral blood monocytes were prepared from the blood of healthy human volunteers collected in EDTA blood tubes. Monocytes were isolated by serial gradient centrifugation and washing to produce a pure population of cells. Lipopolysacharide (LPS) was then added to the cell suspension in tissue culture and this was incubated for 4-12 hours at 37 degrees centigrade. Test mixtures were then added followed by the addition of the P2X₇ receptor agonist BzATP. Test mixtures can comprise of vehicle as control, a P2X₇ receptor antagonist, or a combination of a P2X₇ receptor antagonist together with methotrexate. After incubation, samples of cell supernatants were transferred to a 96-well plate for subsequent cytokine and mediator measurements. The formation of inflammatory mediators was measured in the cell supernatants by specific ELISA assays for the cytokines IL-1, IL-18, TNFα and for other mediators including PGE2, NO and matrix metalloproteinases (MMPs). The levels of mediators released in the presence of a P2X₇ receptor antagonist alone, or in the presence of a combination of a P2X₇ receptor antagonist with methotrexate were determined. The effects produced by a P2X₇ antagonist alone and in combination with methotrexate were then compared. Statistically significant levels of inhibitory activity against a single mediator (IL-1 or TNFα) or on multiple mediators by P2X₇ antagonist/methotrexate combinations in comparison to that achieved by a P2X₇ antagonist alone is an indicator for increased efficacy in the treatment of disease.

### EXAMPLE 3

### Assessment of Anti-Inflammatory Activity of Methotrexate/P2X₇ Antagonist Combinations in Rat Streptococcal Cell Wall-Induced Arthritis¹

Streptococcal cell wall (SCW)-induced arthritis was induced in the left ankle of female Lewis rats. Animals were sensitised by intra-articular injection of 5 μg (in 20 μL) SCW (Lee Laboratories) into the left ankle. Ankle swelling was assessed 3 days after injection and non-responders (animals with no apparent ankle swelling) were rejected. Responding animals were randomly allocated to the test groups.

Arthritis was induced 21 days after sensitisation by intravenous (iv) injection of SCW (100 μg in 500 μL saline). Animals were monitored and assessed on a daily basis through to termination 6 days after induction. The rats were housed on sawdust and provided with food and water ad libitum.

Oral dosing suspensions were in 1% (w/v) methylcellulose in deionised water and were freshly prepared on a daily basis. Compounds were administered by oral (4 mL/kg) prophylactic dosing, commencing 1 day prior to induction of arthritis through to termination on day 6 post-induction. The P2X₇ antagonist 1, was dosed at 30 mg/kg (bid) and the Methotrexate dosed at 0.3 mg/kg (bid).

Ankle diameters were measured with vernier callipers on a daily basis from day-1. Mechanical thresholds were assessed using von Frey filaments on days-1, 1, 3 and 5. The filaments were applied in increasing weights to the ankle region on the footpad of both feet. The first filament to induce a withdrawal response was considered to be the threshold.

Effects on ankle swelling and mechanical threshold were calculated on an area under the curve (AUC) basis, as the sum of the differences from individual day-1 values. Data analysis was by one-way ANOVA followed by Dunnett's test (ankle diameter) or Dunn's test (von Frey threshold) on the raw data (GraphPad Instat).

The left hind limbs were taken and X-rays of the tibio-tarsal compartment examined and scored (blinded) for radiologically evident lesions. Tissues were then processed for histopathologial assessment. Data analysis was by a non-parametric one-way analysis of variance (ANOVA), followed by Kruskal-Wallis post-test.

1. Experimental procedure based on that described by Carlson R P, Jacobsen P B; ‘Comparison of adjuvant and streptococcal cell wall-induced arthritis in the rat’ in Morgan D W, Marshall L A, editors; In Vivo Models of Inflammation. Basel: Birkhauser Verlag; 1999.

1. A pharmaceutical composition comprising, in admixture, a first active ingredient which is a P2X₇ receptor antagonist and which P2X₇ receptor antagonist is an adamantyl derivative, and a second active ingredient which is N-[4-[[(2,4-diamino-6-pteridinyl)methyl]methylamino]benzoyl]-L-glutamic acid or a pharmaceutically acceptable derivative thereof.

2. A composition according to claim 1, wherein the P2X₇ receptor antagonist is a compound of formula 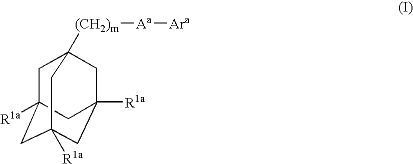
wherein m represents 1, 2 or 3;
each R^{1a} independently represents a hydrogen or halogen atom;
A^{a} represents C(O)NH or NHC(O);
Ar^{a} represents a group 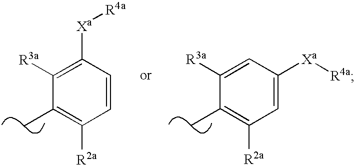
X^{a} represents a bond, an oxygen atom or a group CO, (CH₂)₁₋₆, CH═, (CH₂)₁₋₆O, O(CH₂)₁₋₆, O(CH₂)₂₋₆O, O(CH₂)₂₋₃O(CH₂)₁₋₃, CR′(OH), (CH₂)₁₋₃O(CH₂)₁₋₃, (CH₂)₁₋₃O(CH₂)₂₋₃O, NR^{5a}, (CH₂)₁₋₆NR^{5a}, NR^{5a}(CH₂)₁₋₆, (CH₂)₁₋₃NR^{5a}(CH₂)₁₋₃, O(CH₂)₂₋₆NR^{5a}, O(CH₂)₂₋₃NR^{5a}(CH₂)₁₋₃, (CH₂)₁₋₃NR^{5a}(CH₂)₂₋₃O, NR^{5a}(CH₂)₂₋₆O, NR^{5a}(CH₂)₂₋₃O(CH₂)₁₋₃, CONR^{5a}, NR^{5a}CO, S(O)ₙ, S(O)ₙCH₂, CH₂S(O)ₙ, SO₂NR^{5a} or NR^{5a}SO₂;
n is 0, 1 or 2;
R′ represents a hydrogen atom or a C₁-C₆ alkyl group;
one of R^{2a} and R^{3a} represents a halogen, cyano, nitro, amino, hydroxyl, or a group selected from (i) C₁-C₆ alkyl optionally substituted by at least one C₃-C₆ cycloalkyl, (ii) C₃-C₈ cycloalkyl, (iii) C₁-C₆ alkyloxy optionally substituted by at least one C₃-C₆ cycloalkyl, and (iv) C₃-C₈ cycloalkyloxy, each of these groups being optionally substituted by one or more fluorine atoms, and the other of R^{2a} and R^{3a} represents a hydrogen or halogen atom;
either R^{4a} represents a 3- to 9-membered saturated or unsaturated aliphatic heterocyclic ring system containing one or two nitrogen atoms and optionally an oxygen atom, the heterocyclic ring system being optionally substituted by one or more substituents independently selected from fluorine atoms, hydroxyl, carboxyl, cyano, C₁-C₆alkyl, C₁-C₆ hydroxyalkyl, —NR^{6a}R^{7a}, —(CH₂)ᵣNR^{6a}R^{7a} and —CONR^{6a}R^{7a},
or R^{4a} represents a 3- to 8-membered saturated carbocyclic ring system substituted by one or more substituents independently selected from —NR^{6a}R^{7a}, —(CH₂)ᵣNR^{6a}R^{7a} and —CONR^{6a}R^{7a}, the ring system being optionally further substituted by one or more substituents independently selected from fluorine atoms, hydroxyl and C₁-C₆ alkyl;
r is 1, 2, 3, 4, 5 or 6;
R^{5a} represents a hydrogen atom or a C₁-C₆ alkyl or C₃-C₈ cycloalkyl group;
R^{6a} and R^{7a} each independently represent a hydrogen atom or a C₁-C₆ alkyl, C₂-C₆ hydroxyalkyl or C₃-C₈ cycloalkyl group, or R^{6a} and R^{7a} together with the nitrogen atom to which they are attached form a 3- to 8-membered saturated heterocyclic ring;
with the provisos that,
(a) when A^{a} represents C(O)NH and R^{4a} represents an unsubstituted 3- to 8-membered saturated aliphatic heterocyclic ring system containing one nitrogen atom, then X^{a} is other than a bond, and
(b) when A^{a} represents C(O)NH and X^{a} represents a group (CH₂)₁₋₆ or O(CH₂)₁₋₆, then R^{4a} does not represent an unsubstituted imidazolyl, unsubstituted morpholinyl, unsubstituted piperidinyl or unsubstituted pyrrolidinyl group, and
(c) when A^{a} represents NHC(O) and R^{4a} represents an unsubstituted 3- to 8-membered saturated aliphatic heterocyclic ring system containing one nitrogen atom, then X^{a} is other than a bond, and
(d) when A^{a} represents NHC(O) and X^{a} represents O(CH₂)₁₋₆, NH(CH₂)₁₋₆ or SCH₂, then R^{4a} does not represent an unsubstituted 1-piperidinyl or unsubstituted 1-pyrrolidinyl group, and
(e) when A^{a} represents NHC(O) and X^{a} represents O(CH₂)₂₋₃NH(CH₂)₂, then R^{4a} does not represent an imidazolyl group;
or a pharmaceutically acceptable salt or solvate thereof.

3. A composition according to claim 1, wherein the P2X₇ receptor antagonist is a compound of formula 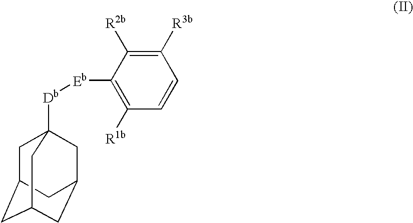
wherein D^{b} represents CH₂ or CH₂CH₂;
E^{b} represents C(O)NH or NHC(O);
R^{1b} and R^{2b} each independently represent a hydrogen or halogen atom, or an amino, nitro, C₁-C₆ alkyl or trifluoromethyl group;
R^{3b} represents a group of formula 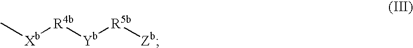
X^{b} represents an oxygen or sulphur atom or a group NH, SO or SO₂;
Y^{b} represents an oxygen or sulphur atom or a group NR^{11b}, SO or SO₂;
Z^{b} represents a group —OH, —SH, —CO₂H, C₁-C₆ alkoxy, C₁-C₆ alkylthio, C₁-C₆-alkylsulphinyl, C₁-C₆-alkylsulphonyl, —NR^{6b}R^{7b}, C(O)NR^{8b}R^{9b}, imidazolyl, 1-methylimidazolyl, —N(R^{10b})C(O)—C₁-C₆ alkyl, C₁-C₆ alkylcarbonyloxy, C₁-C₆ alkoxycarbonyloxy, —OC(O)NR^{12b}R^{13b}, —OCH₂OC(O)R^{14b}, —OCH₂OC(O)OR^{15b} or —OC(O)OCH₂OR^{16b};
R^{4b} represents a C₂-C₆ alkyl group;
R^{5b} represents a C₁-C₆ alkyl group;
R^{6b}, R^{7b}, R^{8b}, R^{9b}, R^{10b}, R^{12b} and R^{13b} each independently represent a hydrogen atom, or a C₁-C₆ alkyl group optionally substituted by at least one hydroxyl group;
R^{11b} represents a hydrogen atom, or a C₁-C₆ alkyl group optionally substituted by at least one substituent independently selected from hydroxyl and C₁-C₆ alkoxy; and
R^{14b}, R^{15b} and R^{16b} each independently represent a C₁-C₆ alkyl group;
with the provisos that (i) when E^{b} represents NHC(O), X^{b} represents O, S or NH and Y^{b} represents O, then Z^{b} represents —NR^{6b}R^{7b} where R^{6b} represents a hydrogen atom and R^{7b} represents either a hydrogen atom or a C₁-C₆ alkyl group substituted by at least one hydroxyl group, and (ii) when E^{b} represents NHC(O), X^{b} represents O, S or NH, Y represents NH and R^{5b} represents CH₂CH₂, then Z^{b} is not —OH or imidazolyl;
or a pharmaceutically acceptable salt or solvate thereof.

4. A composition according to claim 1, wherein the P2X₇ receptor antagonist is a compound of formula 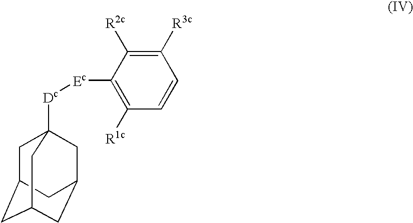
wherein D^{c} represents CH₂ or CH₂CH₂;
E^{c} represents C(O)NH or NHC(O);
R^{1c} and R^{2c} each independently represent hydrogen, halogen, amino, nitro, C₁-C₆ alkyl or trifluoromethyl, but R^{1c} and R^{2c} may not both simultaneously represent hydrogen;
R^{3c} represents a group of formula 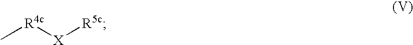
R^{4c} represents a C₁-C₆ alkyl group;
X^{c} represents an oxygen or sulphur atom or a group NR^{13c}, SO or SO₂;
R^{5c} represents hydrogen, or R^{5c} represents C₁-C₆ alkyl or C₂-C₆ alkenyl, each of which may be optionally substituted by at least one substituent selected from halogen, hydroxyl, (di)-C₁-C₆-alkylamino, —Y^{c}—R^{6c}, 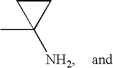
a 5- or 6-membered heteroaromatic ring comprising from 1 to 4 heteroatoms independently selected from nitrogen, oxygen and sulphur which heteroaromatic ring may itself be optionally substituted by at least one substituent selected from halogen, hydroxyl and C₁-C₆ alkyl;
Y represents an oxygen or sulphur atom or a group NH, SO or SO₂;
R^{6c} represents a group —R^{7c}Z^{c} where R^{7c} represents a C₂-C₆ alkyl group and Z^{c} represents an —OH, —CO₂H, —NR^{8c}R^{9c}, —C(O)NR^{10c}R^{11c} or —N(R^{12c})C(O)—C₁-C₆ alkyl group, and, in the case where Y^{c} represents an oxygen or sulphur atom or a group NH, R^{6c} additionally represents hydrogen, C₁-C₆ alkyl, C₁-C₆ alkylcarbonyl, C₁-C₆ alkoxycarbonyl, —C(O)NR^{14c}R^{15c}, —CH₂OC(O)R^{16c}, —CH₂OC(O)OR^{17c} or —C(O)OCH₂OR^{18c};
R^{8c}, R^{9c}, R^{10c}, R^{11c} and R^{12c} each independently represent a hydrogen atom or a C₁-C₆ alkyl group;
R^{13c} represents hydrogen, C₃-C₈ cycloalkyl, C₃-C₈ cycloalkylmethyl, or R^{13c} represents a C₁-C₆ alkyl group optionally substituted by at least one substituent selected from hydroxyl and C₁-C₆ alkoxy; and
R^{14c}, R^{15c}, R^{16c}, R^{17c} and R^{18c} each independently represent a C₁-C₆ alkyl group;
with the proviso that when E^{c} is C(O)NH, X^{c} is O, NH, or N(C₁-C₆ alkyl), then R^{5c} is other than a hydrogen atom or an unsubstituted C₁-C₆ alkyl group;
or a pharmaceutically acceptable salt or solvate thereof.

5. A composition according to claim 1, wherein the P2X₇ receptor antagonist is a compound of formula 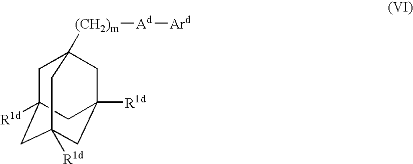
wherein m represents 1, 2 or 3;
each R^{1d} independently represents a hydrogen or halogen atom;
A^{d} represents C(O)NH or NHC(O);
Ar^{d} represents a group 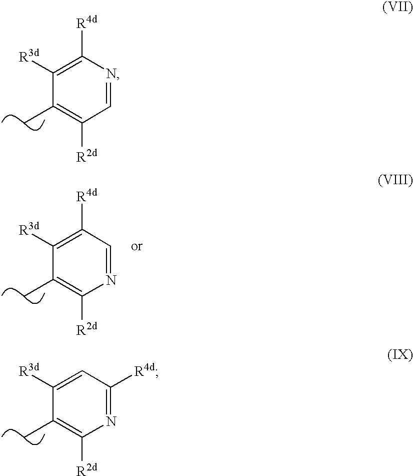
one of R^{2d} and R^{3d} represents halogen, nitro, amino, hydroxyl, or a group selected from (i) C₁-C₆ alkyl optionally substituted by at least one halogen atom, (ii) C₃-C₈ cycloalkyl, (iii) C₁-C₆ alkoxy optionally substituted by at least one halogen atom, and (iv) C₃-C₈ cycloalkyloxy, and the other of R^{2d} and R^{3d} represents a hydrogen or halogen atom;
R^{4d} represents a group 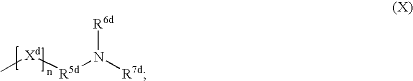
X^{d} represents an oxygen or sulphur atom or a group >N—R^{8d};
n is 0 or 1;
R^{5d} represents a C₁-C₅ alkyl group which may be optionally substituted by at least one substituent selected from hydroxyl, halogen and C₁-C₆ alkoxy;
R^{6d} and R^{7d} each independently represent a hydrogen atom, C₁-C₆ alkyl (optionally substituted by at least one substituent selected from hydroxyl, halogen, C₁-C₆ alkoxy, and (di)-C₁-C₄ alkylamino (itself optionally substituted by at least one hydroxyl group)), or C₃-C₈ cycloalkyl (optionally substituted by at least one substituent selected from hydroxyl, halogen and C₁-C₆ alkoxy); and
R^{8d} represents a hydrogen atom or a C₁-C₅ alkyl group which may be optionally substituted by at least one substituent selected from hydroxyl, halogen and C₁-C₆ alkoxy;
with the provisos that:
when n is 0, then A^{d} is NHC(O), and
when n is 1, X^{d} represents oxygen and A^{d} is C(O)NH, then R^{6d} and R^{7d} do not both simultaneously represent a hydrogen atom or do not both simultaneously represent an unsubstituted C₁-C₆ alkyl, or when one of R^{6d} and R^{7d} represents a hydrogen atom, then the other of R^{6d} and R^{7d} does not represent an unsubstituted C₁-C₆ alkyl; and
when n is 1, X^{d} is oxygen, sulphur or >NH and A^{d} is NHC(O), then R^{6d} and R^{7d} do not both simultaneously represent a hydrogen atom or do not both simultaneously represent an unsubstituted C₁-C₆ alkyl, or when one of R^{6d} and R^{7d} represents a hydrogen atom, then the other of R^{6d} and R^{7d} does not represent an unsubstituted C₁-C₆ alkyl or —CH₂CH₂OH;
or a pharmaceutically acceptable salt or solvate thereof.

6. A composition according to claim 1, wherein the P2X₇ receptor antagonist is a compound of formula 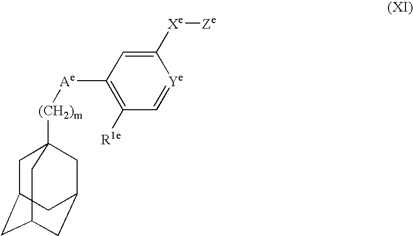
wherein m represents 1, 2 or 3;
A^{e} represents C(O)NH or NHC(O);
Y^{e} represents N or CH;
X^{e} represents a bond, CO, (CH₂)₁₋₆, O(CH₂)₁₋₆, (CH₂)₁₋₆NH(CH₂)₁₋₆, (CH₂)₁₋₆O(CH₂)₁₋₆, NH(CH₂)₁₋₆;
Z^{e} represents NR^{2e}R^{3e};
R^{1e} represents halogen, cyano, nitro, amino, hydroxyl, C₁-C₆ alkyl or C₃-C₈ cycloalkyl, which alkyl or cycloalkyl group can be optionally substituted by one or more fluorine atoms;
R^{2e} and R^{3e} each independently represent a hydrogen atom, C₁-C₆ alkyl or C₃-C₈ cycloalkyl, which alkyl or cycloalkyl group can be optionally substituted by one or more groups selected from hydroxyl, halogen or C₁-C₆ alkoxy,
or R^{2e} and R^{3e} together with the nitrogen atom to which they are attached form a 3- to 9-membered saturated mono- or bicyclic heterocyclic ring comprising from 1 to 2 nitrogen atoms and optionally an oxygen atom, which heterocyclic ring can be optionally substituted by one or more groups selected from hydroxyl, halogen or C₁-C₆ alkoxy;
or a pharmaceutically acceptable salt or solvate thereof.

7. A composition according to claim 1, wherein the P2X₇ receptor antagonist is:
2-Chloro-5-[[2-(2-hydroxy-ethylamino)-ethylamino]-methyl]-N-(tricyclo[3.3.1.1^{3,7}]dec-1-ylmethyl)-benzamide,
2-Chloro-5-[3-[(3-hydroxypropyl)amino]propyl]-N-(tricyclo[3.3.1.1]dec-1-ylmethyl)-benzamide,
(R)-2-Chloro-5-[3-[(2-hydroxy-1-methylethyl)amino]propyl]-N-(tricyclo[3.3.1.1^{3,7}]dec-1-ylmethyl)-benzamide,
2-Chloro-5-[[2-[(2-hydroxyethyl)amino]ethoxy]methyl]-N-(tricyclo[3.3.1.1^{3,7}]dec-1-ylmethyl)-benzamide,
2-Chloro-5-[3-[3-(methylamino)propoxy]propyl]-N-(tricyclo[3.3.1.1^{3,7}]dec-1-ylmethyl)benzamide,
2-Chloro-5-[3-(3-hydroxy-propylamino)-propoxy]-N-(tricyclo[3.3.1.1^{3,7}]dec-1-ylmethyl)-benzamide,
2-Chloro-5-[2-(3-hydroxypropylamino)ethylamino]-N-(tricyclo[3.3.1.1^{3,7}]dec-1-ylmethyl)-benzamide,
2-Chloro-5-[2-(3-hydroxypropylsulfonyl)ethoxy]-N-(tricyclo[3.3.1.1^{3,7}]dec-1-ylmethyl)-benzamide,
2-Chloro-5-[2-[2-[(2-hydroxyethyl)amino]ethoxy]ethoxy]-N-(tricyclo[3.3.1.1^{3,7}]dec-1-ylmethyl)-benzamide,
2-Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide,
2-Chloro-5-piperazin-1-ylmethyl-N-(tricyclo[3.3.1.1]dec-1-ylmethyl)-benzamide,
2-Chloro-5-(4-piperidinyloxy)-N-(tricyclo[3.3.1.1^{3,7}]dec-1-ylmethyl)-benzamide,
2-Chloro-5-(2,5-diazabicyclo[2.2.1]hept-2-ylmethyl)-N-(tricyclo[3.3.1.1]dec-1-ylmethyl)-benzamide,
2-Chloro-5-(piperidin-4-ylsulfinyl)-N-(tricyclo[3.3.1.1^{3,7}]dec-1-ylmethyl)-benzamide,
5-Chloro-2-[3-[(3-hydroxypropyl)amino]propyl]-N-(tricyclo[3.3.1.1^{3,7}]dec-1-ylmethyl)-4-pyridinecarboxamide,
2-Chloro-5-[3-[[(1R)-2-hydroxy-1-methylethyl]amino]propyl]-N-(tricyclo[3.3.1.1^{3,7}]dec-1-ylmethyl)-3-pyridinecarboxamide,
5-Chloro-2-[3-(ethylamino)propyl]-N-(tricyclo[3.3.1.1^{3,7}]dec-1-ylmethyl)-4-pyridinecarboxamide,
5-Chloro-2-[3-[(2-hydroxyethyl)amino]propyl]-N-(tricyclo[3.3.1.1^{3,7}]dec-1-ylmethyl)-4-pyridinecarboxamide,
5-Chloro-2-[3-[[(2S)-2-hydroxypropyl]amino]propyl]-N-(tricyclo[3.3.1.1^{3,7}]dec-1-ylmethyl)-4-pyridinecarboxamide,
N-[2-Methyl-5-(9-oxa-3,7-diazabicyclo[3.3.1]non-3-ylcarbonyl)phenyl]-tricyclo[3.3.1.1^{3,7}]decane-1-acetamide,
or a pharmaceutically acceptable salt or solvate of any one thereof.

8. A composition according to claim 1, wherein the second active ingredient is N-[4-[[(2,4-diamino-6-pteridinyl)methyl]methylamino]benzoyl]-L-glutamic acid.

9. A composition according to claim 1 which is formulated for oral administration.

10. A process for the preparation of a pharmaceutical composition as defined in claim 1 which comprises mixing the first active ingredient with the second active ingredient.

11-12. (canceled)

13. A method of treating an inflammatory disorder which comprises administering a therapeutically effective amount of a pharmaceutical composition as defined in claim 1 to a patient in need thereof.

14. A method according to claim 13, wherein the inflammatory disorder is rheumatoid arthritis.

15. A method of treating a patient comprising administering simultaneously, sequentially, or separately a therapeutically effective amount of a pharmaceutical product comprising, in combination, a preparation of a first active ingredient which is a P2X₇ receptor antagonist and which P2X₇ receptor antagonist is an adamantyl derivative, and a preparation of a second active ingredient which is N-[4-[[(2,4-diamino-6-pteridinyl)methyl]methylamino]benzoyl]-L-glutamic acid or a pharmaceutically acceptable derivative thereof.

16. A kit comprising a preparation of a first active ingredient which is a P2X₇ receptor antagonist and which P2X₇ receptor antagonist is an adamantyl derivative, a preparation of a second active ingredient which is N-[4-[[(2,4-diamino-6-pteridinyl)methyl]methylamino]benzoyl]-L-glutamic acid or a pharmaceutically acceptable derivative thereof, and instructions for the simultaneous, sequential or separate administration of the preparations to a patient in need thereof.

17. The method of claim 15, wherein the patient is treated for an inflammatory disorder.

18. The method of claim 17, wherein the inflammatory disorder is rheumatoid arthritis.
